(19) United States
(12) Patent Application Publication
(10) Pub. No.: US 2024/0378516 A1
(43) Pub. Date: Nov. 14, 2024

(54) SYSTEMS AND METHODS FOR INTELLIGENT SYSTEM RESOURCE ALLOCATION

(71) Applicant: Optum Services (Ireland) Limited, Dublin (IE)

(72) Inventors: Conor John WALDRON, Dublin (IE); Breanndan O’CONCHUIR, Warrington (GB); Michael J. McCARTHY, Dublin (IE); Daniel KELLY, Dublin (IE)

(21) Appl. No.: 18/629,150

(22) Filed: Apr. 8, 2024

Related U.S. Application Data
(60) Provisional application No. 63/501,429, filed on May 11, 2023.

(51) Int. Cl. G06Q 10/0631; G16H 40/00

(52) U.S. Cl. CPC G06Q 10/0631; G16H 40/00

(57) ABSTRACT

Systems and methods are disclosed for allocating system resources. One or more processors may receive a plurality of system data sets and a plurality of user data sets. The one or more processors may determine one or more target systems by applying one or more filters to the plurality of system data sets and one or more target users associated. One or more processors may determine one or more target user data sets associated with each of the one or more target users. One or more processors may apply a machine-learning model to the one or more target user data sets to generate a user-level score. One or more processors may generate a system-level score for each of the one or more target systems associated with the one or more target users. One or more processors may initiate performance of one or more actions.

### CROSS-REFERENCE TO RELATED APPLICATION(S)

This application claims the benefit of priority of U.S. Provisional Patent Application Ser. No. 63/501,429, file May 11, 2023, the disclosure of which is herein incorporated by reference in its entirety.

### TECHNICAL FIELD

The present disclosure relates generally to data analytics. More particularly, the present disclosure relates to adaptation of machine learning techniques for modeling predicted changes in regimen adherence to inform resource allocation.

### BACKGROUND

In the context of service industry resource management, the strategic distribution of limited assets such as human assets is impactful for ensuring effective adoption and adherence to protocols. However, this distribution often follows ad-hoc procedures that overlook predictive analytics based on user data, such as the likelihood to change protocols or the misapplication of protocols due to gaps in user data. This approach leads to computational inefficiencies related to the use of assets, resulting in concerns over achievement of desired outcomes and efficiency metrics. Various strategies have been devised to enhance such efficiencies of resource allocation, including demand forecasting, scheduling adjustments, and the deployment of digital assistance platforms. However, these methods suffer from one or more issues.

For instance, drawbacks of existing methods include inaccurate predictions and failure to target outputs based on user data, which may be indicative of a likelihood of a protocol change. The existing methods primarily react to past data and subjective understanding of user data rather than preemptively identifying and engaging with user data indicative of protocol changes or the misapplication of protocols due to gaps in user data. Predictive models currently in use often rely on generalized data, which may not accurately capture the nuanced factors contributing to risk of user protocol switching. Even if such nuanced factors are included in generalized data, generalized data typically includes a subset of data that causes the generalized data to be noisy, which leads to inefficient processing due to processing of unnecessary data.

Therefore, there is a need for a more sophisticated and accurate approach that utilizes user data to inform predictive analytics related to gaps in user data to inform the allocation of assets.

This disclosure is directed to addressing the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure addresses the technical problem(s) described above or elsewhere in the present disclosure and improves the state of data incident response techniques.

In some aspects, the techniques described herein relate to a computer-implemented method including: receiving, by one or more processors, a plurality of system data sets associated with a plurality of respective systems; receiving, by the one or more processors, a plurality of user data sets associated with a plurality of respective users, each user associated with one or more of the plurality of systems; determining, by the one or more processors and from among the plurality of systems, one or more target systems by applying one or more filters to the plurality of system data sets; determining, by the one or more processors and from among the plurality of users, one or more target users associated with each of the one or more target systems; determining, by the one or more processors and from among the plurality of user data sets, one or more target user data sets associated with each of the one or more target users; applying, by the one or more processors, a machine-learning model to the one or more target user data sets to generate a user-level score for each of the one or more target users; generating, by the one or more processors and based on the user-level score for each of the one or more target users, a system-level score for each of the one or more target systems associated with the one or more target users; and initiating, by the one or more processors, performance of one or more actions in response to the generating.

In some aspects, the techniques described herein relate to a system including memory and one or more processors communicatively coupled to the memory, the one or more processors configured to: receive a plurality of system data sets associated with a plurality of respective systems; receive a plurality of user data sets associated with a plurality of respective users, each user associated with one or more of the plurality of systems; determine, from among the plurality of systems, one or more target systems by applying one or more filters to the plurality of system data sets; determine, from among the plurality of users, one or more target users associated with each of the one or more target systems; determine, from among the plurality of user data sets, one or more target user data sets associated with each of the one or more target users; apply a machine-learning model to the one or more target user data sets to generate a user-level score for each of the one or more target users; generate, based on the user-level score for each of the one or more target users, a system-level score for each of the one or more target systems associated with the one or more target users; and initiate performance of one or more actions in response to the generating.

In some aspects, the techniques described herein relate to one or more non-transitory computer-readable storage media including instructions that, when executed by one or more processors, cause the one or more processors to: receive a plurality of system data sets associated with a plurality of respective systems; receive a plurality of user data sets associated with a plurality of respective users, each user associated with one or more of the plurality of systems; determine, from among the plurality of systems, one or more target systems by applying one or more filters to the plurality of system data sets; determine, from among the plurality of users, one or more target users associated with each of the one or more target systems; determine, from among the plurality of user data sets, one or more target user data sets associated with each of the one or more target users; apply a machine-learning model to the one or more target user data sets to generate a user-level score for each of the one or more target users; generate, based on the user-level score for each of the one or more target users, a system-level score for each of the one or more target systems associated with the one or more target users; and initiate performance of one or more actions in response to the generating.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1A is a diagram showing an example of a system environment, according to some embodiments of the disclosure.

FIG. 1B is a diagram of example components of a resource allocation platform, according to some embodiments of the disclosure.

FIG. 1C is a diagram of example components of a machine-learning module, according to some embodiments of the disclosure.

FIG. 2 is a flowchart showing a method for system resource allocation, according to some embodiments of the disclosure.

FIG. 3 is a diagram depicting example input and output data to one or more machine-learning models is provided.

FIG. 4 shows an example machine-learning training flow chart, according to some embodiments of the disclosure.

FIG. 5 illustrates an implementation of a computer system that executes techniques presented herein, according to some embodiments of the disclosure.

### DETAILED DESCRIPTION

In some embodiments, the present disclosure pertains to the application of artificial intelligence (AI) in formulating resource allocation strategies. This disclosure encompasses systems and methods for identifying, using machine-learning techniques, entities (e.g., healthcare providers) with high potential for using a specific product (e.g., drug, pharmaceutical product, medical equipment, etc.). Specifically, it introduces frameworks and methodologies for modeling the likelihood of entities to switch their members from competitor products to their own products, thereby optimizing the allocation of resources.

As an example, traditional resource allocation techniques in a healthcare setting often fall short in effectively identifying and prioritizing healthcare providers who are most likely to prescribe a specific drug. These conventional practices lack in offering tailored analysis of healthcare providers' prescribing behaviors, understanding local market dynamics, ensuring alignment with strategic growth objectives, and utilizing advanced predictive analytics to accurately identify opportunities for increasing prescription rates.

To address these concerns, the current disclosure introduces a centralized platform for comprehensive analysis and prioritization of healthcare providers based on their likelihood to switch prescriptions from a competitor's drug to the sponsoring company's drug. The platform receives data related to systems (e.g., prescribing doctors or medical systems) and related to users of those systems (e.g., patients). The users are each associated with a system. The platform then applies one or more filters to the systems to identify target systems, such as systems within a certain geographical region. For the users of the target systems, the platform applies a filter, this filter being based on whether each user is currently taking a prescription for a certain medication and/or whether the user has one or more diagnosed or undiagnosed conditions. Then, data associated with each user is applied to one or more machine-learning models, which generate a user-level score for each user. The platform then utilizes the user-level score for each user within a target system to determine an overall score, or a system-level score, for the target system. After performing this for a variety of systems, the platform produces system-level scores which indicate a comparative metric across each system. Based on the scores, and the comparative scores across each system, the platform initiates one or more action, such as allocating resources to high-scoring systems. This platform leverages predictive Al models, such as the RETAIN model, which utilizes healthcare data including diagnosis codes, procedure codes, prescription codes, and recent medical lab values to generate predictive scores for healthcare providers.

The technical improvements and advantages of this invention include improving the efficiency and effectiveness of resource allocation in systems (e.g., healthcare provider systems), enabling personalized healthcare strategies through the identification of target systems and users (e.g., patients), and enhancing predictive analytics capabilities with machine learning to forecast healthcare needs and treatment outcomes. These benefits contribute to a more informed and strategic approach to healthcare resource management, potentially leading to better patient care, optimized use and/or distribution of resources, and more efficient healthcare systems.

Moreover, the solutions outlined herein leverage detailed medical history and prescription data of patients treated by individual healthcare providers and propose provider-specific strategies within the context of broader market dynamics. The framework and methodology also entail continuous monitoring of healthcare providers' prescribing behaviors and market dynamics, with subsequent adjustments, updates, and model retraining to accommodate shifts in healthcare provider behavior or market conditions. This leads to greater efficacy in targeting strategies, enhanced efficiency in resource allocation for pharmaceutical sales, and a reduction in the complexity of managing pharmaceutical resource allocation efforts. The technical advancements and additional enhancements facilitated by this disclosure are elucidated in detail throughout the document. It should be clear to those skilled in the art that the technical improvements provided herein extend beyond those explicitly mentioned, encompassing further advancements in the field of data analytics, predictive analytics, and artificial intelligence.

The technical improvements and advantages discussed above are not the sole improvements and advantages, and additional technical improvements and advantages will be discussed in the following sections. Further, based on the present disclosure, other technical improvements and advantages will be apparent to one of ordinary skill in the art.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

Various non-limiting embodiments of the present disclosure will now be described to provide an overall understanding of the principles of the structure, function, and use of systems and methods disclosed herein for resource allocation.

Reference to any particular activity is provided in this disclosure only for convenience and not intended to limit the disclosure. A person of ordinary skill in the art would recognize that the concepts underlying the disclosed devices and methods may be utilized in any suitable activity. For example, while the present disclosure is in the context of resource allocation in a healthcare setting, one of ordinary skill would understand the applicability of the described systems and methods to similar tasks in a variety of contexts or environments. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Training the machine-learning model may include one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-Prototypes or K-Means may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. After training the machine-learning mode, the machine-learning model may be deployed in a computer application for use on new input data that it has not been trained on previously.

As used herein, “system data” broadly refers to a comprehensive spectrum of information and metrics generated, collected, or utilized by various systems within the scope of healthcare provider analysis and pharmaceutical resource allocation efforts. This includes, but is not limited to, healthcare provider identifiers, prescription histories, patient demographic information, treatment outcomes, and market share analytics. Additionally, system data encompasses electronic health records (EHR), pharmacy dispensing data, claims data from insurers, and any other datasets relevant to understanding healthcare provider behaviors, patient care patterns, and competitive landscape dynamics. Furthermore, the term extends to include data infrastructure components and analytical tools involved in the processing, analysis, and transmission of this information, such as database management systems, data processing algorithms, machine learning models, and communication networks.

As used herein, “user data” comprehensively encompasses all forms of information and records related to individuals who interact with or are impacted by the healthcare and pharmaceutical sectors. This includes, but is not limited to, patient health records, prescription records, diagnostic codes, procedure histories, and personal demographic details such as age, gender, and geographical location. User data also covers patient-reported outcomes, adherence to medication regimes, and responses to treatment, providing a holistic view of patient health and behavior. Additionally, this term extends to encompass data collected from healthcare providers, including prescribing patterns, preferences for specific medications, and feedback on drug efficacy and safety. The scope of user data further includes engagement metrics from digital health tools, patient portals, and telehealth platforms, capturing the interactions between patients and healthcare systems in a digital context. This data is pivotal in creating patient-centric models for pharmaceutical resource allocation.

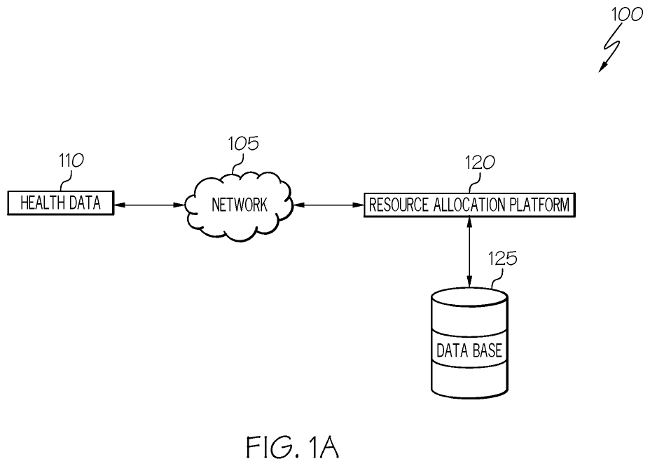

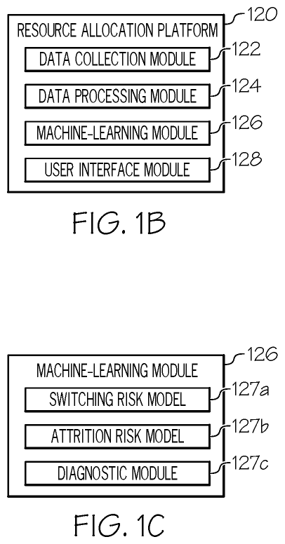

FIG. 1A is a diagram showing an example of a system environment, according to some embodiments of the disclosure. The depicted environment, labeled as environment 100, is presented in line with a particular embodiment of this disclosure. Environment 100 includes a communication infrastructure known as network 105, which is connected to health data 110, and further integrates with a resource allocation platform 120 that incorporates a database 125.

In one embodiment, various components within environment 100 interact via network 105. Network 105 enables communication between resource allocation platform 120 and other systems and/or data within the environment 100, such as health data 110. Health data 110 may contain data, data entries, and/or data objects relevant to members, claims, health indicators, or the like associated with the environment 100. Network 105 can comprise various types of networks, including but not limited to data networks, wireless networks, telephony networks, or any combination thereof, facilitating robust and secure data flow across environment 100. Within environment 100, any of these components, including health data 110, resource allocation platform 120, and nodes 130, may communicate with one another based on established access permissions, which may be dictated by one or more access permission associated with user 115.

Any of the health data 110 associated with the resource allocation platform 120 may contain a diverse collection of structured and unstructured data pertinent to transactions and operational processes within the healthcare environment. In some embodiments, this data, organized into one or more data objects, spans a variety of dimensions including medical claims, patient diagnoses, prescription data, place of service data, and other relevant clinical and administrative data. This extensive repository, which includes medical histories, treatment plans, prescription records, and compliance statuses, is housed in storage solutions that may range from local to cloud-based data storage systems, ensuring secure storage and accessibility for ongoing processing and analytical evaluation.

The database 125 may support the storage and retrieval of various types of data related to one or more data sets and/or data objects, such as medical claims, patient diagnoses, prescription data, place of service data, and other relevant clinical and administrative data. This database may store metadata and operational data about one or more entities represented in these data sets, as well as any information received from the resource allocation platform 120. The database may comprise one or more systems, including but not limited to a relational database management system (RDBMS), a NoSQL database, or a graph database, tailored to meet the specific needs and use cases within the healthcare environment.

In one embodiment, database 125 can be any type of database system, such as relational, hierarchical, object-oriented, etc., where data is systematically organized in tables, lookup tables, or other appropriate structures. Database 125 is responsible for storing and facilitating access to data utilized by resource allocation platform 120, encompassing information related to transaction and operational logs as well as outputs generated by the platform. It is capable of storing a wide variety of information to assist in the management, security, and operation of the environment.

In one embodiment, database 125 includes a machine learning-based training database that delineates relationships, associations, and connections between input parameters from healthcare provider and patient data and output parameters representing various metrics for pharmaceutical resource allocation and healthcare provider targeting. For instance, the training database might incorporate machine learning algorithms designed to learn mappings between data inputs and outputs such as healthcare provider prescribing potential, patient adherence risk scores, market penetration indicators, prescription trend forecasts, and the like. This training database is periodically updated to reflect additional insights gained through ongoing machine learning processes, thereby enhancing the accuracy and effectiveness of predictive models in identifying high-potential healthcare providers and optimizing pharmaceutical sales strategies.

Resource allocation platform 120 communicates with other components within network 105 using established or emerging protocols. These protocols facilitate interactions between various system elements and define the conventions for creating, sending, and interpreting data exchanged across communication links. They function across different layers, ranging from the generation of physical signals to the recognition of specific software applications engaged in data analysis, prediction, and resource allocation decisions. This multilayered communication approach ensures seamless integration and coordination between the resource allocation platform, data collection and processing modules, and machine learning algorithms, thereby enabling efficient and targeted deployment of pharmaceutical sales resources.

Communications between the various components of the networks are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers.

In operation, environment 100 serves as a platform for processing and analyzing healthcare provider and patient data within the pharmaceutical industry, utilizing techniques such as data analytics, artificial intelligence, and database management. For instance, environment 100 facilitates the generation of insights, metrics, and data objects from various datasets, including healthcare provider prescribing behaviors and patient health records, according to predefined criteria or multiple parameters.

To execute these functions, the resource allocation platform 120 employs methods such as one or more machine-learning models, such as switching risk model 127a, attrition risk model 127b, and/or diagnostic model 127c, which interpret healthcare data to identify patterns, trends, and opportunities for pharmaceutical resource allocation. Additionally, the resource allocation platform 120 leverages the data collection module 122 and the data processing module 124 to aggregate and refine healthcare data for further analysis.

For efficient data storage and access, the database 125 archives metadata associated with the healthcare data, including information about data origins, types, and structures. This database also retains details on the insights generated by the resource allocation platform 120, such as healthcare provider potential scores, patient adherence indicators, and statistical data.

Beyond healthcare data analysis, environment 100 supports a range of applications, including data visualization, search functionalities, and predictive modeling. For example, it enables users on one or more user devices to query healthcare data for specific metrics that meet certain criteria or to visualize healthcare statistics through dynamic graphs and charts.

FIG. 1B is a diagram of example components of a resource allocation platform, according to some embodiments of the disclosure. This figure shows that the resource allocation platform 120, as part of environment 100, has the functionality to analyze various datasets, such as healthcare provider and patient data, and generate data objects, including insights and metrics relevant to pharmaceutical resource allocation. The terms “component” or “module” within this context refer to both hardware and software implemented by a processor or similar technology. Specifically, the resource allocation platform 120 is equipped with modules for collecting, processing, analyzing healthcare data, and generating data objects. These include the data collection module 122, the data processing module 124, the machine-learning module 126, and the user interface module 128. The design allows for flexibility in how these modules are organized, with the possibility of integrating their functions into fewer modules or distributing them across different modules with similar capabilities.

In certain embodiments, the data collection module 122 of the resource allocation platform 120 is responsible for gathering data from various sources and formats during the operation of environment 100. This module is capable of handling a wide range of data types, including, but not limited to, electronic health records, prescription data, patient demographics, treatment outcomes, and healthcare provider prescribing patterns. Additionally, it can process proprietary or generated data like patient adherence models, healthcare provider potential scores, and market analysis outputs.

The data is ingested into the system via multiple pathways, thereby providing flexibility in the collection mechanism. Specifically, one pathway includes an Application Programming Interface (API) that establishes a secure communication channel for automated data transfer between the data collection module 122 and external healthcare data sources, thus facilitating real-time or batch-based data acquisition. Another pathway allows for manual input by authorized users via a dedicated user interface, where such input can be executed through file uploads or direct data entry into predefined fields. Additionally, data intake can be accomplished through third-party integrations, middleware, or direct database queries that serve to populate the database 125. The data collection module 122 further incorporates data validation and integrity checks to ensure the consistency and reliability of the ingested data. By offering a plurality of data intake methodologies, the data collection module 122 ensures robust and comprehensive data assimilation for downstream processing.

In some embodiments, the data processing module 124 of the resource allocation platform 120 is involved in processing and preparing healthcare provider and patient data for further analysis by the machine-learning module 126. The data processing module 124 undertakes the cleaning of data, elimination of irrelevant or redundant information, and conversion of the data into a format suitable for analysis by the machine-learning module 126. It is designed to enhance the initial data collection by transforming the raw, diverse healthcare and pharmaceutical data into a unified, standardized format for accurate and efficient analysis downstream. Specifically, the data processing module 124 employs a series of algorithms for data normalization, addressing inconsistencies in data formats, units, or terminologies from various healthcare data sources.

The data processing module 124 further integrates error-handling mechanisms to detect and correct possible data inaccuracies or anomalies within the healthcare data. These mechanisms can include rule-based checks, probabilistic data matching, or data imputation techniques, all aimed at maintaining data quality and integrity for healthcare analytics. Additionally, the data processing module 124 may feature parallel processing capabilities to manage multiple healthcare data streams simultaneously, enhancing the timeliness and efficiency of data throughput. This attribute is especially beneficial for handling large datasets or facilitating real-time analytics, where rapid processing of healthcare provider and patient data is critical.

Upon receiving the processed data from the data processing module 124, the machine-learning module 126 applies algorithms and models to generate one or more data objects, including insights and metrics relevant to healthcare provider targeting and pharmaceutical resource allocation strategies. The machine-learning module 126 utilizes a variety of algorithms and machine-learning models to achieve this, engaging in the computational analysis of the ingested healthcare and pharmaceutical data. Utilizing the machine-learning models such as the switching risk model 127a, the attrition risk model 127b, and the diagnostic model 127c as part of its analytical framework, the machine-learning module 126 employs a mix of algorithmic and machine-learning methodologies to produce metrics and data objects based on the input data. These metrics and data objects provide quantifiable insights into the prescribing behaviors, patient health trends, and market opportunities within the pharmaceutical industry.

After generating the data objects, including insights and metrics, a user interface presented on a user device through the user interface module 128 displays the results to the user in a timely manner. This interface offers an interactive and intuitive platform for users to view, analyze, or act upon the generated insights. It also allows users to provide feedback or input additional parameters to refine the analysis or adjust the models within the resource allocation platform 120 accordingly. The user interface module 128 is configured to facilitate user interaction, enabling the input of parameters through an interactive interface, thereby enhancing the decision-making process for pharmaceutical resource allocation and healthcare provider engagement strategies.

FIG. 1C is a diagram of example components of a machine-learning module, according to some embodiments of the disclosure. FIG. 1C provides a more detailed view of the machine-learning module and its relationship with the switching risk model 127a, the attrition risk model 127b, and the diagnostic model 127c within the resource allocation platform 120. The machine-learning module 126 is designed to harness advanced analytical capabilities to process, analyze, and generate predictions from vast datasets related to healthcare provider behaviors, patient health trends, and other relevant healthcare and pharmaceutical data. This module acts as the core analytical engine of the resource allocation platform, integrating various predictive models to support comprehensive resource allocation strategies.

The machine-learning module 126 is equipped with sophisticated algorithms that enable it to perform a wide range of functions, from data preprocessing and feature extraction to the application of complex predictive models. It is structured to facilitate the seamless integration and operation of specific models tailored to address distinct aspects of healthcare and pharmaceutical resource allocation. These models include the switching risk model 127a, designed to predict the likelihood of healthcare providers switching patients from one drug to another; the attrition risk model 127b, aimed at identifying the risk of patients discontinuing treatment of a target protocol; and the diagnostic model 127c, which forecasts the likelihood of patients being diagnosed with certain conditions that may require specific pharmaceutical interventions.

In some embodiments, the switching risk model 127a, as part of the machine-learning module 126, is tasked with analyzing and understanding the dynamics of healthcare provider prescribing behaviors and patient medication adherence patterns within the pharmaceutical industry. In particular, the prediction is the risk of patients switching from a non-target medication to a target medication. This model processes data pertaining to healthcare provider characteristics, patient demographics, treatment histories, and other relevant information to generate predictions about the likelihood of healthcare providers switching patients from one drug to another. By analyzing patterns in prescribing behaviors, patient responses to treatments, and market trends, the switching risk model 127a provides insights into factors influencing drug switching decisions.

To accomplish these tasks, the model employs advanced machine learning algorithms to sift through vast quantities of healthcare and pharmaceutical data, identifying patterns and relationships that may not be immediately apparent. This enables the resource allocation platform to offer insights into the prescribing tendencies of healthcare providers, facilitating more informed decision-making regarding resource allocation and engagement strategies.

The switching risk model 127a continuously learns from new data and evolving healthcare industry conditions, with the machine-learning module 126 updating the training of the model in response to received data about healthcare provider behaviors and patient treatment outcomes. This ensures that the model's predictions and recommendations remain relevant and accurate, providing a dynamic tool for the proactive management of pharmaceutical resources.

In some embodiments, the switching risk model 127a within the machine-learning module 126 may be characterized as a multi-modal model. This model may incorporate and process data from diverse formats, including, but not limited to, textual data, numerical data, and structured data formats. Specifically, the switching risk model 127a is capable of analyzing a wide range of healthcare data, enhancing its understanding and analysis of factors influencing drug switching behaviors.

Furthermore, the switching risk model 127a may employ advanced algorithms capable of processing diverse data types, such as machine learning techniques suitable for analyzing structured healthcare data. This capability may be integrated with the analysis of textual and numerical data using techniques appropriate for those formats. It will be appreciated that various forms of machine-learning models may be utilized, depending on the specific requirements of the context.

The training and continuous updating of the switching risk model 127a involve the iterative refinement of its parameters based on feedback from the system's performance and the emergence of new data. This process may include retraining the model with updated healthcare provider and patient data, reflecting changes in prescribing behaviors or treatment outcomes, as well as incorporating new insights from ongoing healthcare industry research. The dynamic updating mechanism ensures that the switching risk model 127a remains attuned to the evolving state of the healthcare and pharmaceutical industries, enabling it to provide relevant and timely insights for optimizing resource allocation strategies.

In some embodiments, the attrition risk model 127b, integrated within the machine-learning module 126, is designed to assess and predict the risk of patients discontinuing their prescribed medications within the pharmaceutical context. In particular, the prediction is the risk of patients switching from a target medication to a non-target medication. This model leverages data on patient adherence history, demographic information, treatment side effects, and healthcare provider engagement levels, among other factors, to forecast patient attrition risks. By analyzing historical adherence patterns, patient-healthcare provider interactions, and medication efficacy reports, the attrition risk model 127b builds associations between one or more factors and/or inputs and patient continuation with one or more treatment plans.

To achieve its objectives, the model utilizes sophisticated machine learning algorithms to navigate through the complex landscape of healthcare and patient data, unveiling subtle patterns and correlations that inform attrition risk. This capacity empowers the resource allocation platform to discern and strategize interventions tailored to enhancing patient adherence, thereby informing broader resource allocation and patient support initiatives.

The attrition risk model 127b is dynamically refined through continuous learning from new patient data and evolving treatment paradigms, with the machine-learning module 126 periodically updating the model's training. This iterative learning process ensures the attrition risk predictions and intervention recommendations maintain their relevance and precision, serving as an adaptive resource in managing patient adherence challenges.

In some embodiments, the attrition risk model 127b is depicted as a multi-modal model within the machine-learning module 126, capable of assimilating and analyzing data from varied sources and formats. This includes structured patient records, treatment regimen details, and patient-reported outcome measures, providing a comprehensive dataset for analysis. The multi-modal approach of the attrition risk model 127b enables a holistic view of patient adherence factors, leveraging the strengths of diverse data types to yield a nuanced understanding of attrition risks.

Moreover, the attrition risk model 127b employs advanced analytical algorithms tailored to the specific nature of the healthcare data it processes, such as predictive modeling techniques and statistical analysis tools. These algorithms are chosen and refined to suit the complexity and specificity of the data involved, ensuring that the attrition risk assessments are both accurate and actionable.

The training and ongoing refinement of the attrition risk model 127b involve adjusting its parameters in response to new insights from patient adherence trends and emerging healthcare research. This includes incorporating updated patient data that reflect shifts in adherence patterns, as well as integrating new findings from adherence studies. Such dynamic updating enhances the model's capability to deliver current and contextually relevant guidance for mitigating the risk of patient attrition, thereby optimizing patient outcomes and resource allocation within the pharmaceutical landscape.

In some embodiments, the diagnostic model 127c, integrated within the machine-learning module 126, is designed to assess and predict the risk of patients being diagnosed with specific conditions within the pharmaceutical context. This model leverages data on patient health history, demographic information, symptom presentations, and healthcare provider engagement levels, among other factors, to forecast diagnostic risks. By analyzing historical health patterns, patient-healthcare provider interactions, and clinical outcomes reports, the diagnostic model 127c builds associations between one or more factors and/or inputs and patient diagnosis likelihoods.

To achieve its objectives, the model utilizes sophisticated machine learning algorithms to navigate through the complex landscape of healthcare and patient data, unveiling subtle patterns and correlations that inform diagnostic risk. This capacity empowers the resource allocation platform to discern and strategize interventions tailored to enhancing disease detection and management, thereby informing broader resource allocation and patient support initiatives.

The diagnostic model 127c is dynamically refined through continuous learning from new patient data and evolving healthcare paradigms, with the machine-learning module 126 periodically updating the model's training. This iterative learning process ensures the diagnostic predictions and intervention recommendations maintain their relevance and precision, serving as an adaptive resource in managing healthcare diagnostics and patient care.

In certain embodiments, the diagnostic model 127c is depicted as a multi-modal model within the machine-learning module 126, capable of assimilating and analyzing data from varied sources and formats. This includes structured patient records, clinical examination results, and patient-reported symptoms, providing a comprehensive dataset for analysis. The multi-modal approach of the diagnostic model 127c enables a holistic view of diagnostic factors, leveraging the strengths of diverse data types to yield a nuanced understanding of diagnostic risks.

Moreover, the diagnostic model 127c employs advanced analytical algorithms tailored to the specific nature of the healthcare data it processes, such as predictive modeling techniques and statistical analysis tools. These algorithms are chosen and refined to suit the complexity and specificity of the data involved, ensuring that the diagnostic assessments are both accurate and actionable.

The training and ongoing refinement of the diagnostic model 127c involve adjusting its parameters in response to new insights from patient health trends and emerging medical research. This includes incorporating updated patient data that reflect shifts in disease patterns, as well as integrating new findings from diagnostic studies. Such dynamic updating enhances the model's capability to deliver current and contextually relevant guidance for improving diagnostic accuracy and efficiency, thereby optimizing patient outcomes and resource allocation within the pharmaceutical landscape.

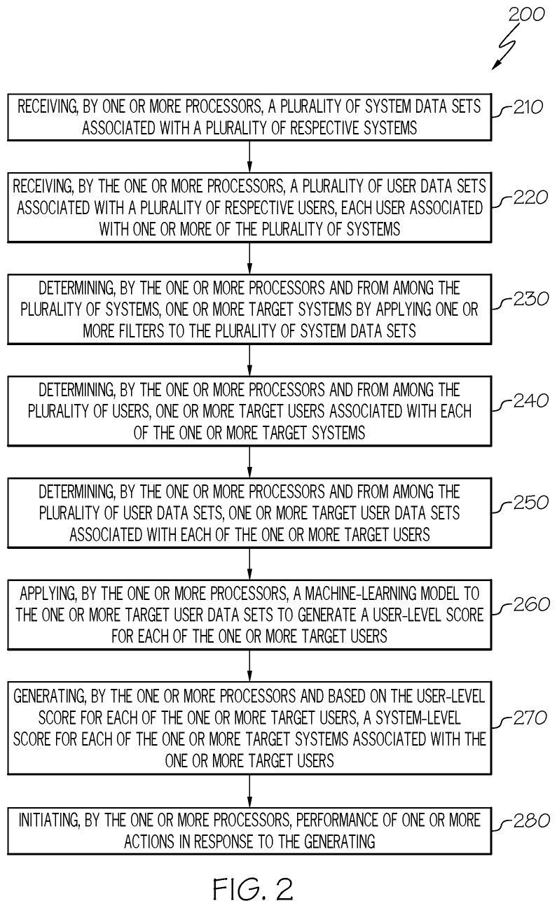

FIG. 2 is a flowchart showing a method 200 for system resource allocation, according to some embodiments of the disclosure. The steps in the method 200 are performed by the resource allocation platform 120, or the individual components therein discussed in the preceding sections. At step 210, the method may include receiving, by one or more processors or the resource allocation platform 120, a plurality of system data sets associated with a plurality of respective systems (e.g., healthcare provider systems). These system data sets may encompass comprehensive collections of data elements that are pertinent to the operation, performance, and management of the respective systems within the context of healthcare provider and pharmaceutical analytics. Each system data set may include, but is not limited to, healthcare provider prescribing data, patient health records, pharmaceutical sales data, market share information, and other relevant data that can inform resource allocation strategies. The reception of these data sets by the processors signifies the initial phase of a data processing and analysis workflow designed to optimize pharmaceutical resource allocation and engagement efforts based on predictive modeling and analysis.

In some embodiments, at step 220, the method further includes receiving, by the one or more processors, a plurality of user data sets associated with a plurality of respective users (e.g., patients), each user associated with one or more of the plurality of systems. These user data sets may contain a range of information pertinent to individual health profiles, treatment histories, demographic details, and interaction records with the healthcare system. Included within these data sets could be medical diagnosis records, prescription data, treatment response records, patient feedback, and adherence metrics, among other data types. The reception of these data sets enables the processors to aggregate and analyze detailed insights into patient behaviors, health outcomes, and preferences.

In some embodiments, in relation to step 220, each of the plurality of user data sets includes a time-series data set. This time-series data set comprises a chronological sequence of entries, with each entry corresponding to one or more protocols associated with the user and arranged according to the order in which the one or more protocols were employed or recorded. The protocols in this context may encompass a wide range of healthcare and treatment-related activities, such as medication prescriptions, diagnostic tests, treatment procedures, and patient-reported outcomes. The chronological arrangement of these entries allows for a dynamic and temporal analysis of patient interactions with the healthcare system, enabling the identification of patterns, trends, and potential outcomes over time. This structured approach to organizing user data facilitates the application of advanced analytical techniques, including machine learning models, to predict future healthcare needs, protocol adherence, and potential adjustments to treatment plans. By leveraging the temporal aspect of patient data, healthcare providers and pharmaceutical strategies can be tailored to improve patient care and optimize resource allocation.

In some embodiments, at step 230, the method includes determining, by the one or more processors, one or more target systems (e.g., target healthcare provider systems) from among the plurality of systems by applying one or more filters to the plurality of system data sets. This step involves the analytical processing of system data sets to identify specific systems that meet predetermined criteria or thresholds, which are relevant to the objectives of healthcare provider targeting and resource allocation strategies. The filters applied may be based on various factors, such as system performance metrics, geographic location, market share data, or specific healthcare provider practices and behaviors. This selective filtering process aims to narrow down the broader set of systems to those with the highest potential for achieving the desired outcomes, such as improved patient care, increased prescription rates, or enhanced market penetration.

In some embodiments, the filtering process in step 230 may also relate to identifying systems based on a target protocol, such as a specific drug or drug category, and determining if the providers within those systems prescribe the target drug or fall within the desired drug category. This involves analyzing the system data sets to extract information on healthcare provider prescribing behaviors, including the types of medications prescribed, frequencies of prescriptions, and alignment with particular pharmaceutical products or categories. The filter criteria may be designed to isolate systems where providers are either already prescribing the target drug or are deemed likely candidates for adopting the target drug into their prescribing practices based on related prescribing patterns or patient demographics.

In some embodiments, at step 240, the method includes determining, by the one or more processors and from among the plurality of users, one or more target users (e.g., target patients) associated with each of the one or more target systems. This determination process involves analyzing user data sets to identify individuals who meet specific criteria making them relevant targets within the context of the identified systems. Criteria for targeting users may include, but are not limited to, their health conditions, prescription histories, adherence to treatments, potential responsiveness to certain pharmaceutical products or treatment protocols, relative regional drug coverage, relative regional market share, or the like.

The identification of target users is based on their association with the target systems previously identified, ensuring a strategic alignment between user needs and system capabilities. This step may involve the application of filters or algorithms designed to highlight users who, for example, are currently prescribed a competitor's drug or a target drug but have characteristics suggesting they could benefit from switching to the target drug, or who have conditions that are underdiagnosed or undertreated within the target systems, or to highlight systems which may be of particular value, such as systems located in high prevalence areas of a target condition or systems located in areas where market coverage is low relative to one or more target drugs.

In some embodiments, at step 250, the method includes determining, by the one or more processors and from among the plurality of user data sets, one or more target user data sets associated with each of the one or more target users. This operation involves the data processing module 124 executing selection and extraction algorithms, configured to isolate specific data sets corresponding to the identified target users based on predefined attributes. These attributes could include, but are not limited to, diagnostic codes, prescription records, and other pertinent healthcare interactions that align with the target user profiles. Utilizing query mechanisms and data filtering technologies, the data processing module 124 navigates through the user data sets. This ensures the association of relevant data subsets with each target user, facilitated by the computational power and analytical capabilities of the one or more processors integrated within the resource allocation platform 120. The result of this step is a meticulously curated collection of target user data sets, each with information specific to the individual target users.

In some embodiments, at step 260, the method includes applying, by the one or more processors, a machine-learning model to the one or more target user data sets to generate a user-level score for each of the one or more target users. At step 260, the machine-learning module 126 within the resource allocation platform 120 executes this process, utilizing the switching risk model 127a, the attrition risk model 127b, or the diagnostic model 127c, depending on the specific objectives of the analysis. The application of the machine-learning model involves processing each target user data set through the model's algorithms to assess and quantify factors such as the likelihood of switching medications, risk of treatment discontinuation, or probability of diagnosis with a specific condition.

The model analyzes patterns, trends, and correlations within the data, extracting features that contribute to the score's computation. These features might include, but are not limited to, historical medication adherence levels, frequency of healthcare provider interactions, diagnostic test results, and other relevant health indicators. The output, a user-level score, represents a quantified assessment of the target user's position relative to the analysis objective, such as their risk profile or potential for treatment optimization.

In some embodiments, at step 260, the method includes the machine-learning module 126 applying the switching risk model 127a to the one or more target user data sets to generate a user-level score for each of the one or more target users. The user-level score, in this context, is associated with the risk or likelihood of the user switching from an alternative protocol to a target protocol. Specifically, the switching risk model 127a intakes data associated with various factors within the target user data sets, such as historical prescription data, patient health records, and other relevant indicators that might influence a healthcare provider's decision to transition a patient from one medication to another or indicate a patent's likelihood to switch for one or more other reason, such as ineffectiveness, side effects, price, or the like. This comprehensive analysis facilitates the prediction of which users are more likely to switch protocols, enabling tailored interventions aimed at encouraging or facilitating this transition.

In the context of the switching risk model 127a, the machine-learning module 126 is specifically configured to discern and quantify the associations between user data sets and the likelihood of a user transitioning from a first protocol to a second protocol. The training of model 127a involves a meticulous process where the first protocol is identified and associated with one or more objects, which could be medications, treatment plans, or healthcare practices. This identification serves as a baseline for comparison and analysis within the user data sets.

The model's training further encompasses the examination of user data sets linked to individual users. These data sets are scrutinized for evidence indicating whether the associated user has previously adhered to the first protocol before switching to the second protocol or has remained on the first protocol without making a transition.

Adjustment of the model's parameters is a continuous process informed by the insights gained from the analysis of user data sets. Parameters are fine-tuned to enhance the model's accuracy in predicting protocol switches. This adjustment is predicated on the complex interplay of variables within the user data sets, including, but not limited to, adherence rates, patient outcomes, healthcare provider recommendations, and other relevant factors that may influence a user's decision to switch protocols. Through this iterative training and adjustment and/or modification process, model 127a becomes increasingly adept at identifying users who are likely candidates for transitioning from one protocol to another, thus facilitating interventions and resource allocation aimed at enhancing patient adherence and retention.

In some embodiments, when the attrition risk model 127b is applied, the process similarly generates a user-level score for each target user, but with a focus on the risk of the user discontinuing use of a target protocol in favor of an alternative protocol. This model intakes data associated with adherence patterns, patient-reported outcomes, various factors within the target user data sets, such as historical prescription data, patient health records, and other relevant indicators that might influence use of the target medication, among other factors. The attrition risk score thus reflects the likelihood of a patient stopping their current treatment, providing valuable insights for developing strategies to improve patient engagement, adherence, and overall satisfaction with their treatment plan.

In the context of the attrition risk model 127b, the machine-learning module 126 is trained to identify and quantify the associations between user data sets and the likelihood of a user discontinuing a first protocol in favor of an alternative protocol or ceasing treatment altogether. The model's training further includes an identification of user data sets corresponding to individual users and identification of whether the associated user has discontinued the first protocol in favor of an alternative protocol or has stopped following the first protocol without adopting a new one. This identification maps out the attrition patterns and understanding the factors contributing to users' decisions to discontinue a given treatment protocol.

Adjustment of the model's parameters is a continuous process informed by the insights gained from the analysis of user data sets. Parameters are refined, adjusted, or modified to improve the model's predictive performance regarding user attrition. This refinement relies on a nuanced understanding of the variables captured in the user data sets, such as treatment efficacy, side effects, patient satisfaction, and interactions with healthcare providers, which could influence a user's decision to abandon their current treatment protocol. Through this training and parameter adjustment process, model 127b evolves to more accurately predict which users are at risk of discontinuing their treatment, thus facilitating interventions and resource allocation aimed at enhancing patient adherence and retention.

In some embodiments, when employing the diagnostic model 127c, the machine-learning module 126 calculates a user-level score indicating the likelihood of a user having one or more target conditions. This model intakes data through user data sets for symptoms, diagnostic codes, lab results, and other health indicators pertinent to the one or more conditions of interest. The resulting diagnostic likelihood score aids healthcare providers and pharmaceutical companies in identifying patients who may be undiagnosed or at high risk for certain conditions, thereby directing appropriate resources or identifying systems which a high number of undiagnosed target members.

In some embodiments, inputs to a machine learning model, such as the switching risk model 127a, the attrition risk model 127b, or the diagnostic model 127c within the machine-learning module 126, may comprise time-series data sets. These time-series data sets consist of chronological sequences of entries that detail user interactions with various healthcare protocols over time. Prior to their utilization within the machine learning model, these time-series data sets undergo a process of alignment to meet the input requirements of the model. This alignment process involves formatting, normalizing, and potentially transforming the data to ensure compatibility with the model's architecture. Once appropriately processed, the time-series data set is inputted into the machine learning model, facilitating the generation of a user-level score for each user. This score reflects the model's assessment of the user's likelihood to switch from one protocol to another, the risk of discontinuing a current treatment, or the probability of having or developing a specific health condition, based on the temporal patterns and trends identified within the processed time-series data.

In some embodiments, at step 270, the method 200 includes generating, by the one or more processors and based on the user-level score for each of the one or more target users, a system-level score for each of the one or more target systems associated with the one or more target users. This generation is based on the user-level score derived for each of the target users from preceding steps, utilizing models such as the switching risk model 127a, the attrition risk model 127b, or the diagnostic model 127c within the machine-learning module 126. The system-level score aggregates the insights gleaned from individual user-level scores to provide a composite assessment reflective of the overall potential or risk profile of each target system.

In some embodiments, the aggregation process involves the application algorithms that weigh the individual user scores, possibly factoring in the number of users, the distribution of scores, and specific user attributes that might amplify or mitigate the system's overall score. This could include considerations such as the prevalence of a particular condition within a system, the propensity of users within a system to switch medications, or the adherence patterns observed among users associated with a system. The resulting system-level score serves as a metric of evaluation for healthcare providers, healthcare systems, or pharmaceutical entities to identify which systems represent the highest priority for intervention, resource allocation, or further analysis.

In some embodiments, the method involves normalizing the scores based on the number of users associated with each system, executed by the one or more processors. Following the aggregation of weighted user-level scores to compute a preliminary system-level score, this normalization step adjusts the aggregated score to accurately represent the system's collective health or risk profile, factoring in the total user count within each system. The normalization is effected by dividing the aggregated weighted score by the number of users in the system or employing statistical normalization methods to standardize scores across systems, ensuring comparability.

In some embodiments, at step 280, the method includes initiating, by the one or more processors, performance of one or more actions in response to the generation of system-level scores or user-level scores, specifically focusing on resource allocation strategies. The actions initiated entail ordering the one or more target systems based on the system score for each of the one or more target systems, enabling a strategic prioritization of resources across the healthcare ecosystem. Additionally, the processors identify a subset of the one or more target systems whose scores surpass a predetermined threshold, earmarking them as high-priority areas for resource distribution.

In some embodiments, the method includes comparing the user-level score against a predetermined threshold. This comparison identifies users who meet or exceed specific criteria that signal a heightened interest, need, or risk within the healthcare system. When a user-level score surpasses this threshold, a flag is applied to the user's data set. The nature of this flag varies depending on the context of the score; it may indicate that the user is not currently on the target drug but is likely to switch, that the user is already on the target drug but presents a risk of attrition, or that the user has a probability above a threshold value of having a target condition. The flag, in some embodiments, serves as a filter on the users associated with the system, thus only factoring in flagged users in the calculation of the system score.

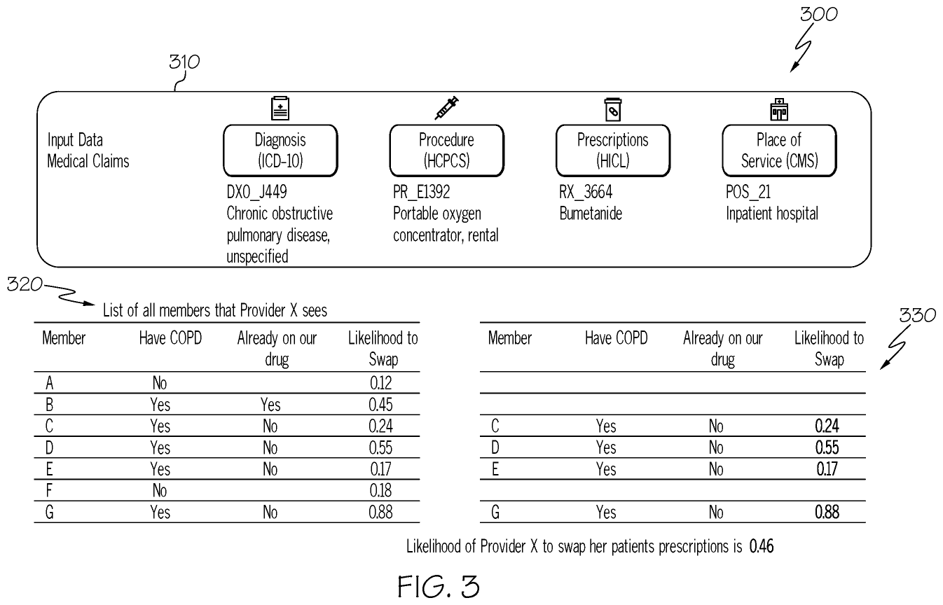

Referring to FIG. 3, a diagram depicting example input and output data to one or more machine-learning model is provided. The diagram include input data, a pre-filtered output, and a filtered output.

The first section 310 of FIG. 3 shows the input data for a machine-learning model, emphasizing the incorporation of user medical data. This data comprises an extensive range of medical information, including but not limited to, medical claims, diagnosis records, procedural details, prescription data, and place of service information. The inclusion of such diverse medical data inputs ensures a holistic view of each user's health status and treatment history, providing a foundation for the predictive analysis performed by the machine-learning model.

The second section 320, FIG. 3 shows a chart exemplifying the output for members associated with one system out of a plurality of systems. For each member, one or more data is displayed, such as the presence of a target condition flag (shown in this example as COPD) indicating whether the member has the target condition, a status indicator for whether the member is currently using the target drug, and a numerical output from the model. This numerical output represents the likelihood of each member switching from their current medication to an alternative drug, offering valuable insights into patient treatment trajectories and potential areas for intervention.

The third section of FIG. 3 shows a chart where a filter is applied to the member data, specifically excluding those members who are already on the target drug. Following the application of this filter, a system score is calculated for the remaining members, reflecting the aggregated likelihood of medication switch within the system. This system score is instrumental in identifying systems with a high potential for successful intervention in terms of encouraging the adoption of the target drug among members not currently receiving it. By focusing on members not on the target medication, the method optimizes resource allocation and intervention strategies to target the most receptive populations, thereby enhancing treatment outcomes and efficiency in healthcare delivery.

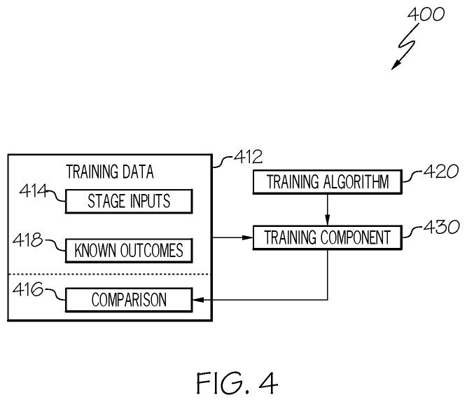

One or more implementations disclosed herein include and/or are implemented using a machine-learning model. For example, one or more of the modules of the resource allocation platform 120 are implemented using a machine-learning model and/or are used to train the machine-learning model. FIG. 4 shows an example machine-learning training flow chart, according to some embodiments of the disclosure. Referring to FIG. 4, a given machine-learning model is trained using the training flow chart 400. The training data 412 includes one or more of stage inputs 414 and the known outcomes 418 related to the machine-learning model to be trained. The stage inputs 414 are from any applicable source including text, visual representations, data, values, comparisons, and stage outputs, e.g., one or more outputs from one or more steps from FIG. 2. The known outcomes 418 are included for the machine-learning models generated based on supervised or semi-supervised training, or can based on known labels, such as topic labels. An unsupervised machine-learning model is not trained using the known outcomes 418. The known outcomes 418 includes known or desired outputs for future inputs similar to or in the same category as the stage inputs 414 that do not have corresponding known outputs.

The training data 412 and a training algorithm 420, e.g., one or more of the modules implemented using the machine-learning model and/or are used to train the machine-learning model, is provided to a training component 430 that applies the training data 412 to the training algorithm 420 to generate the machine-learning model. According to an implementation, the training component 430 is provided comparison results 416 that compare a previous output of the corresponding machine-learning model to apply the previous result to re-train the machine-learning model. The comparison results 416 are used by the training component 430 to update the corresponding machine-learning model. The training algorithm 420 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIG. 2 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

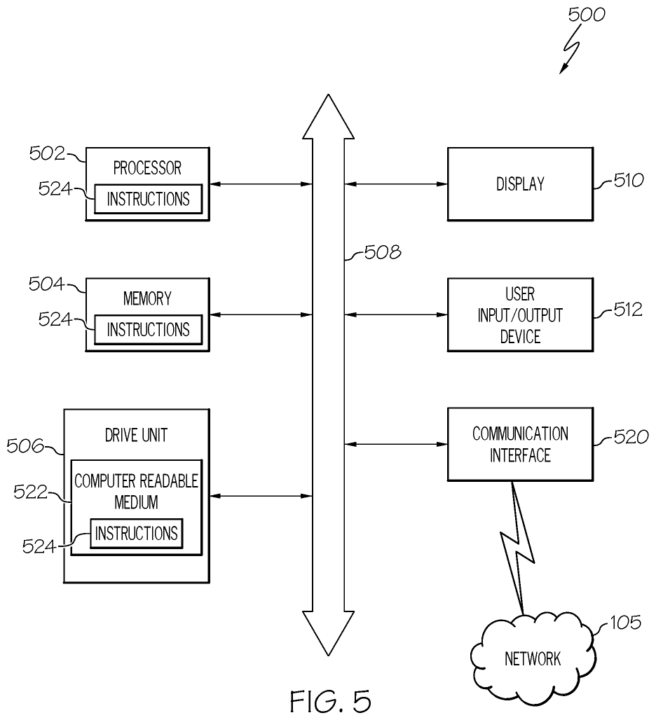

FIG. 5 illustrates an implementation of a computer system that executes techniques presented herein. The computer system 500 includes a set of instructions that are executed to cause the computer system 500 to perform any one or more of the methods or computer based functions disclosed herein. The computer system 500 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 500 operates in the capacity of a server or as a client user computer in a server-client user environment, or as a peer computer system in a peer-to-peer (or distributed) environment. The computer system 500 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 is implemented using electronic devices that provide voice, video, or data communication. Further, while the computer system 500 is illustrated as a single system, the term “system” shall also be taken to include any collection of systems or sub-systems that individually or jointly execute a set, or multiple sets, of instructions to perform one or more computer functions.

As illustrated in FIG. 5, the computer system 500 includes a processor 502, e.g., a central processing unit (CPU), a graphics processing unit (GPU), or both. The processor 502 is a component in a variety of systems. For example, the processor 502 is part of a standard personal computer or a workstation. The processor 502 is one or more processors, digital signal processors, application specific integrated circuits, field programmable gate arrays, servers, networks, digital circuits, analog circuits, combinations thereof, or other now known or later developed devices for analyzing and processing data. The processor 502 implements a software program, such as code generated manually (i.e., programmed).

The computer system 500 includes a memory 504 that communicates via bus 508. The memory 504 is a main memory, a static memory, or a dynamic memory. The memory 504 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts, or tasks illustrated in the figures or described herein are performed by the processor 502 executing the instructions stored in the memory 504. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 500 further includes a display 510, such as a liquid crystal display (LCD), an organic light emitting diode (OLED), a flat panel display, a solid-state display, a cathode ray tube (CRT), a projector, a printer or other now known or later developed display device for outputting determined information. The display 510 acts as an interface for the user to see the functioning of the processor 502, or specifically as an interface with the software stored in the memory 504 or in the drive unit 506.

Additionally or alternatively, the computer system 500 includes an input/output device 512 configured to allow a user to interact with any of the components of the computer system 500. The input/output device 512 is a number pad, a keyboard, a cursor control device, such as a mouse, a joystick, touch screen display, remote control, or any other device operative to interact with the computer system 500.

The computer system 500 also includes the drive unit 506 implemented as a disk or optical drive. The drive unit 506 includes a computer-readable medium 522 in which one or more sets of instructions 524, e.g. software, is embedded. Further, the sets of instructions 524 embodies one or more of the methods or logic as described herein. The sets of instructions 524 resides completely or partially within the memory 504 and/or within the processor 502 during execution by the computer system 500. The memory 504 and the processor 502 also include computer-readable media as discussed above.

In some systems, computer-readable medium 522 includes the set of instructions 524 or receives and executes the set of instructions 524 responsive to a propagated signal so that a device connected to network 105 communicates voice, video, audio, images, or any other data over the network 105. Further, the sets of instructions 524 are transmitted or received over the network 105 via the communication port or interface 520, and/or using the bus 508. The communication port or interface 520 is a part of the processor 502 or is a separate component. The communication port or interface 520 is created in software or is a physical connection in hardware. The communication port or interface 520 is configured to connect with the network 105, external media, the display 510, or any other components in the computer system 500, or combinations thereof. The connection with the network 105 is a physical connection, such as a wired Ethernet connection, or is established wirelessly as discussed below. Likewise, the additional connections with other components of the computer system 500 are physical connections or are established wirelessly. The network 105 alternatively be directly connected to the bus 508.

While the computer-readable medium 522 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 522 is non-transitory, and may be tangible.

The computer-readable medium 522 includes a solid-state memory such as a memory card or other package that houses one or more non-volatile read-only memories. The computer-readable medium 522 is a random-access memory or other volatile re-writable memory. Additionally or alternatively, the computer-readable medium 522 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 500 is connected to the network 105. The network 105 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 105 includes wide area networks (WAN), such as the Internet, local area networks (LAN), campus area networks, metropolitan area networks, a direct connection such as through a Universal Serial Bus (USB) port, or any other networks that allows for data communication. The network 105 is configured to couple one computing device to another computing device to enable communication of data between the devices. The network 105 is generally enabled to employ any form of machine-readable media for communicating information from one device to another. The network 105 includes communication methods by which information travels between computing devices. The network 105 is divided into sub-networks. The sub-networks allow access to all of the other components connected thereto or the sub- networks restrict access between the components. The network 105 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the instructions for carrying out such a method or element of a method forms a means for carrying out the method or element of a method. Furthermore, an element described herein of an apparatus embodiment is an example of a means for carrying out the function performed by the element for the purpose of carrying out the disclosure.

In the description provided herein, numerous specific details are set forth. However, it is understood that embodiments of the disclosure are practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognize that other and further modifications are made thereto without departing from the spirit of the disclosure, and it is intended to claim all such changes and modifications as falling within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects:
Example 1. A computer-implemented method comprising: receiving, by one or more processors, a plurality of system data sets associated with a plurality of respective systems; receiving, by the one or more processors, a plurality of user data sets associated with a plurality of respective users, each user associated with one or more of the plurality of systems; determining, by the one or more processors and from among the plurality of systems, one or more target systems by applying one or more filters to the plurality of system data sets; determining, by the one or more processors and from among the plurality of users, one or more target users associated with each of the one or more target systems; determining, by the one or more processors and from among the plurality of user data sets, one or more target user data sets associated with each of the one or more target users; applying, by the one or more processors, a machine-learning model to the one or more target user data sets to generate a user-level score for each of the one or more target users; generating, by the one or more processors and based on the user-level score for each of the one or more target users, a system-level score for each of the one or more target systems associated with the one or more target users; and initiating, by the one or more processors, performance of one or more actions in response to the generating.
Example 2. The computer-implemented method of example 1, further comprising: comparing, by the one or more processors, the user-level score against a pre-determined threshold, wherein a flag is applied when the user-level score exceeds the pre-determined threshold, and wherein generating the system-level score is based on the applied flag.
Example 3. The computer-implemented method of any of examples 1-2, wherein the machine-learning model is trained to identify associations between a user data set and a likelihood of a user associated with the user data set switching from a first protocol to a second protocol.
Example 4. The computer-implemented method of example 3, wherein the machine-learning model is trained by: identifying a first protocol, the first protocol associated with one or more objects; identifying one or more user data sets associated with one or more users, each of the one or more user data sets including an indication that the associated user a) adhered to the first protocol prior to switching to the second protocol or b) adhered to the first protocol without adhering to the second protocol; and adjusting one or more parameters of the machine-learning model based on the identified one or more user data sets.
Example 5. The computer-implemented method of any of examples 1-4, wherein determining the one or more target users associated with each of the one or more target systems comprises: applying one or more filters to user data sets of users associated with the target system to identify the one or more target user data sets, each of the one or more target user data sets indicating the associated user has one or more conditions.
Example 6. The computer-implemented method of any of examples 1-5, wherein generating the system score comprises applying a weight to each of the one or more target users based on the one or more target user data sets associated with the target user, and aggregating the one or more user-level scores of the one or more target users based on the weight associated with each of the one or more target users.
Example 7. The computer-implemented method of any of examples 1-6, wherein each of the plurality of user data sets includes a time-series data set, the time-series data set comprising a chronological sequence of entries, each entry corresponding to one or more protocols associated with the user and arranged according to an order in which the one or more protocols were employed or recorded.
Example 8. The computer-implemented method of example 7, wherein generating the user-level score comprises: processing the time-series data set to align with an input requirement of the machine-learning model; and inputting the processed time-series data set into the machine-learning model to generate the user-level score for each target user.
Example 9. The computer-implemented method of any of examples 1-8, wherein at least one of the one or more actions comprises: ordering the one or more target systems based on the system score for each of the one or more target systems; identifying a subset of the one or more target systems with a respective system score that exceeds a predetermined first threshold; and presenting, on a graphical user interface, a geographical representation of the subset of the one or more target systems overlaid on a map.
Example 10. A system comprising memory and one or more processors communicatively coupled to the memory, the one or more processors configured to: receive a plurality of system data sets associated with a plurality of respective systems; receive a plurality of user data sets associated with a plurality of respective users, each user associated with one or more of the plurality of systems; determine, from among the plurality of systems, one or more target systems by applying one or more filters to the plurality of system data sets; determine, from among the plurality of users, one or more target users associated with each of the one or more target systems; determine, from among the plurality of user data sets, one or more target user data sets associated with each of the one or more target users; apply a machine-learning model to the one or more target user data sets to generate a user-level score for each of the one or more target users; generate, based on the user-level score for each of the one or more target users, a system-level score for each of the one or more target systems associated with the one or more target users; and initiate performance of one or more actions in response to the generating.
Example 11. The system of example 10, wherein the one or more processors are further configured to: compare the user-level score against a pre-determined threshold, wherein a flag is applied when the user-level score exceeds the pre-determined threshold, and wherein generating the system-level score is based on the applied flag.
Example 12. The system of any of examples 10-11, wherein the machine-learning model is trained to identify associations between a user data set and a likelihood of a user associated with the user data set switching from a first protocol to a second protocol.
Example 13. The system of example 12, wherein the machine-learning model is trained by: identifying a first protocol, the first protocol associated with one or more objects; identifying one or more user data sets associated with one or more users, each of the one or more user data sets including an indication that the associated user a) adhered to the first protocol prior to switching to the second protocol or b) adhered to the first protocol without adhering to the second protocol; and adjusting one or more parameters of the machine-learning model based on the identified one or more user data sets.
Example 14. The system of any of examples 10-13, wherein determining the one or more target users associated with each of the one or more target systems comprises: applying one or more filters to user data sets of users associated with the target system to identify the one or more target user data sets, each of the one or more target user data sets indicating the associated user has one or more conditions.
Example 15. The system of any of examples 10-14, wherein generating the system score comprises applying a weight to each of the one or more target users based on the one or more target user data sets associated with the target user, and aggregating the one or more user-level scores of the one or more target users based on the weight associated with each of the one or more target users.
Example 16. The system of any of examples 10-15, wherein each of the plurality of user data sets includes a time-series data set, the time-series data set comprising a chronological sequence of entries, each entry corresponding to one or more protocols associated with the user and arranged according to an order in which the one or more protocols were employed or recorded.
Example 17. The system of example 16, wherein generating the user-level score comprises: processing the time-series data set to align with an input requirement of the machine-learning model; and inputting the processed time-series data set into the machine-learning model to generate the user-level score for each target user.
Example 18. The system of any of examples 10-17, wherein at least one of the one or more actions comprises: ordering the one or more target systems based on the system score for each of the one or more target systems; identifying a subset of the one or more target systems with a respective system score that exceeds a predetermined first threshold; and presenting, on a graphical user interface, a geographical representation of the subset of the one or more target systems overlaid on a map.
Example 19. One or more non-transitory computer-readable storage media including instructions that, when executed by one or more processors, cause the one or more processors to: receive a plurality of system data sets associated with a plurality of respective systems; receive a plurality of user data sets associated with a plurality of respective users, each user associated with one or more of the plurality of systems; determine, from among the plurality of systems, one or more target systems by applying one or more filters to the plurality of system data sets; determine, from among the plurality of users, one or more target users associated with each of the one or more target systems; determine, from among the plurality of user data sets, one or more target user data sets associated with each of the one or more target users; apply a machine-learning model to the one or more target user data sets to generate a user-level score for each of the one or more target users; generate, based on the user-level score for each of the one or more target users, a system-level score for each of the one or more target systems associated with the one or more target users; and initiate performance of one or more actions in response to the generating.
Example 20. The one or more non-transitory computer-readable storage media of example 19, wherein at least one of the one or more actions comprises: ordering the one or more target systems based on the system score for each of the one or more target systems; identifying a subset of the one or more target systems with a respective system score that exceeds a predetermined first threshold; and presenting, on a graphical user interface, a geographical representation of the subset of the one or more target systems overlaid on a map.
Example 21. The computer-implemented method of any of examples 2-3, wherein the training is performed by the one or more processors.
Example 22. The computer-implemented method of any of examples 2-3, wherein: the one or more processors are included in a first computing entity; and the training is performed by one or more processors included in a second computing entity.

What is claimed is:

1. A computer-implemented method comprising:
receiving, by one or more processors, a plurality of system data sets associated with a plurality of respective systems;
receiving, by the one or more processors, a plurality of user data sets associated with a plurality of respective users, each user associated with one or more of the plurality of systems;
determining, by the one or more processors and from among the plurality of systems, one or more target systems by applying one or more filters to the plurality of system data sets;
determining, by the one or more processors and from among the plurality of users, one or more target users associated with each of the one or more target systems;
determining, by the one or more processors and from among the plurality of user data sets, one or more target user data sets associated with each of the one or more target users;
applying, by the one or more processors, a machine-learning model to the one or more target user data sets to generate a user-level score for each of the one or more target users;
generating, by the one or more processors and based on the user-level score for each of the one or more target users, a system-level score for each of the one or more target systems associated with the one or more target users; and
initiating, by the one or more processors, performance of one or more actions in response to the generating.

2. The computer-implemented method of claim 1, further comprising:
comparing, by the one or more processors, the user-level score against a pre-determined threshold,
wherein a flag is applied when the user-level score exceeds the pre-determined threshold, and
wherein generating the system-level score is based on the applied flag.

3. The computer-implemented method of claim 1, wherein the machine-learning model is trained to identify associations between a user data set and a likelihood of a user associated with the user data set switching from a first protocol to a second protocol.

4. The computer-implemented method of claim 3, wherein the machine-learning model is trained by:
identifying a first protocol, the first protocol associated with one or more objects;
identifying one or more user data sets associated with one or more users, each of the one or more user data sets including an indication that the associated user a) adhered to the first protocol prior to switching to the second protocol or b) adhered to the first protocol without adhering to the second protocol; and
adjusting one or more parameters of the machine-learning model based on the identified one or more user data sets.

5. The computer-implemented method of claim 1, wherein determining the one or more target users associated with each of the one or more target systems comprises:
applying one or more filters to user data sets of users associated with the target system to identify the one or more target user data sets, each of the one or more target user data sets indicating the associated user has one or more conditions.

6. The computer-implemented method of claim 1, wherein generating the system score comprises applying a weight to each of the one or more target users based on the one or more target user data sets associated with the target user, and aggregating the one or more user-level scores of the one or more target users based on the weight associated with each of the one or more target users.

7. The computer-implemented method of claim 1, wherein each of the plurality of user data sets includes a time-series data set, the time-series data set comprising a chronological sequence of entries, each entry corresponding to one or more protocols associated with the user and arranged according to an order in which the one or more protocols were employed or recorded.

8. The computer-implemented method of claim 7, wherein generating the user-level score comprises:
processing the time-series data set to align with an input requirement of the machine-learning model; and
inputting the processed time-series data set into the machine-learning model to generate the user-level score for each target user.

9. The computer-implemented method of claim 1, wherein at least one of the one or more actions comprises:
ordering the one or more target systems based on the system score for each of the one or more target systems;
identifying a subset of the one or more target systems with a respective system score that exceeds a predetermined first threshold; and
presenting, on a graphical user interface, a geographical representation of the subset of the one or more target systems overlaid on a map.

10. A system comprising memory and one or more processors communicatively coupled to the memory, the one or more processors configured to:
receive a plurality of system data sets associated with a plurality of respective systems;
receive a plurality of user data sets associated with a plurality of respective users, each user associated with one or more of the plurality of systems;
determine, from among the plurality of systems, one or more target systems by applying one or more filters to the plurality of system data sets;
determine, from among the plurality of users, one or more target users associated with each of the one or more target systems;
determine, from among the plurality of user data sets, one or more target user data sets associated with each of the one or more target users;
apply a machine-learning model to the one or more target user data sets to generate a user-level score for each of the one or more target users;
generate, based on the user-level score for each of the one or more target users, a system-level score for each of the one or more target systems associated with the one or more target users; and
initiate performance of one or more actions in response to the generating.

11. The system of claim 10, wherein the one or more processors are further configured to:
compare the user-level score against a pre-determined threshold,
wherein a flag is applied when the user-level score exceeds the pre-determined threshold, and
wherein generating the system-level score is based on the applied flag.

12. The system of claim 10, wherein the machine-learning model is trained to identify associations between a user data set and a likelihood of a user associated with the user data set switching from a first protocol to a second protocol.

13. The system of claim 12, wherein the machine-learning model is trained by:
identifying a first protocol, the first protocol associated with one or more objects;
identifying one or more user data sets associated with one or more users, each of the one or more user data sets including an indication that the associated user a) adhered to the first protocol prior to switching to the second protocol or b) adhered to the first protocol without adhering to the second protocol; and
adjusting one or more parameters of the machine-learning model based on the identified one or more user data sets.

14. The system of claim 10, wherein determining the one or more target users associated with each of the one or more target systems comprises:
applying one or more filters to user data sets of users associated with the target system to identify the one or more target user data sets, each of the one or more target user data sets indicating the associated user has one or more conditions.

15. The system of claim 10, wherein generating the system score comprises applying a weight to each of the one or more target users based on the one or more target user data sets associated with the target user, and aggregating the one or more user-level scores of the one or more target users based on the weight associated with each of the one or more target users.

16. The system of claim 10, wherein each of the plurality of user data sets includes a time-series data set, the time-series data set comprising a chronological sequence of entries, each entry corresponding to one or more protocols associated with the user and arranged according to an order in which the one or more protocols were employed or recorded.

17. The system of claim 16, wherein generating the user-level score comprises:
processing the time-series data set to align with an input requirement of the machine-learning model; and
inputting the processed time-series data set into the machine-learning model to generate the user-level score for each target user.

18. The system of claim 10, wherein at least one of the one or more actions comprises:
ordering the one or more target systems based on the system score for each of the one or more target systems;
identifying a subset of the one or more target systems with a respective system score that exceeds a predetermined first threshold; and
presenting, on a graphical user interface, a geographical representation of the subset of the one or more target systems overlaid on a map.

19. One or more non-transitory computer-readable storage media including instructions that, when executed by one or more processors, cause the one or more processors to:
receive a plurality of system data sets associated with a plurality of respective systems;
receive a plurality of user data sets associated with a plurality of respective users, each user associated with one or more of the plurality of systems;
determine, from among the plurality of systems, one or more target systems by applying one or more filters to the plurality of system data sets;
determine, from among the plurality of users, one or more target users associated with each of the one or more target systems;
determine, from among the plurality of user data sets, one or more target user data sets associated with each of the one or more target users;
apply a machine-learning model to the one or more target user data sets to generate a user-level score for each of the one or more target users;
generate, based on the user-level score for each of the one or more target users, a system-level score for each of the one or more target systems associated with the one or more target users; and
initiate performance of one or more actions in response to the generating.

20. The one or more non-transitory computer-readable storage media of claim 19, wherein at least one of the one or more actions comprises:
ordering the one or more target systems based on the system score for each of the one or more target systems;
identifying a subset of the one or more target systems with a respective system score that exceeds a predetermined first threshold; and
presenting, on a graphical user interface, a geographical representation of the subset of the one or more target systems overlaid on a map.
